(19) United States
(12) Patent Application Publication
(10) Pub. No.: US 2014/0011778 A1
(43) Pub. Date: Jan. 9, 2014

(54) 2-METHYLENE-19,23,24-TRINOR-1ALPHA-HYDROXYVITAMIN D3

(71) Applicant: Wisconsin Alumni Research Foundation, Madison, WI (US)

(72) Inventors: Hector F. DeLUCA, Deerfield, WI (US); Margaret Clagett-Dame, Deerfield, WI (US); Lori A. Plum, Arena, WI (US); Agnieszka Glebocka, Madison, WI (US); Rafal Sicinski, Warsaw (PL)

(73) Assignee: Wisconsin Alumni Research Foundation, Madison, WI (US)

(21) Appl. No.: 14/025,488

(22) Filed: Sep. 12, 2013

Related U.S. Application Data
(63) Continuation of application No. 13/592,921, filed on Aug. 23, 2012.
(60) Provisional application No. 61/527,795, filed on Aug. 26, 2011.

(51) Int. Cl. C07C 401/00

(52) U.S. Cl. CPC C07C 401/00
USPC 514/167; 552/653

(57) ABSTRACT

Compounds of Formula I are provided where R¹ and R² are independently selected from H or hydroxy protecting groups. Such compounds may be used in preparing pharmaceutical compositions and are useful in treating a variety of biological conditions.

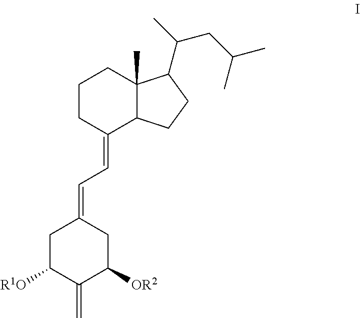

### CROSS-REFERENCE TO RELATED APPLICATIONS

This application is a continuation of U.S. patent application Ser. No. 13/592,921, filed on Aug. 23, 2012, which claims the priority benefit of Provisional Patent Application 61/527,795, filed on Aug. 26, 2011, and is also related to PCT/US2012/052029, filed on Aug. 23, 2012, the contents of all being hereby incorporated by reference.

### FIELD

The present technology relates to vitamin D compounds, and more particularly to diastereomers of 2-methylene-19,23,24-trinor-1α-hydroxyvitamin D₃ and derivatives thereof, and to pharmaceutical compositions that include these compounds. The present technology also relates to the use of these compounds in the treatment of various diseases and in the preparation of medicaments for use in treating various diseases.

### BACKGROUND

The natural hormone, 1α,25-dihydroxyvitamin D₃ (also referred to as 1α,25-dihydroxycholecalciferol and calcitriol) and its analog in the ergosterol series, i.e., 1α,25-dihydroxyvitamin D₂, are known to be highly potent regulators of calcium homeostasis in animals and humans, and their activity in cellular differentiation has also been established, Ostrem et al., Proc. Natl. Acad. Sci. USA, 84, 2610 (1987). Many structural analogs of these metabolites have been prepared and tested, including 1α-hydroxyvitamin D₃, 1α-hydroxyvitamin D₂, various side chain homologated vitamins, and fluorinated analogs. Some of these compounds exhibit an interesting separation of activities in cell differentiation and calcium regulation. This difference in activity may be useful in the treatment of a variety of diseases as renal osteodystrophy, vitamin D-resistant rickets, osteoporosis, psoriasis, and certain malignancies. The structure of 1α,25-dihydroxyvitamin D₃ and the numbering system used to denote the carbon atoms in this compound are shown below.

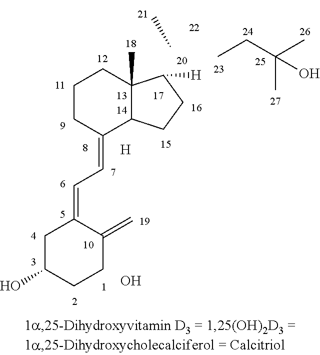

### SUMMARY

The present technology provides diastereomers of 2-methylene-19,23,24-trinor-1α-hydroxyvitamin D₃, including, for example, (20S)-2-methylene-19,23,24-trinor-1α-hydroxyvitamin D₃, (20R)-2-methylene-19,23,24-trinor-1α-hydroxyvitamin D₃, and related compounds, pharmaceutical compositions that include a diastereomer of 2-methylene-19,23,24-trinor-1α-hydroxyvitamin D₃, methods of treating or preventing various disease states using these compounds, and the use of these compounds in the preparation of medicaments for treating or preventing various disease states.

Therefore, in one aspect, the present technology provides a compound having the Formula I shown below

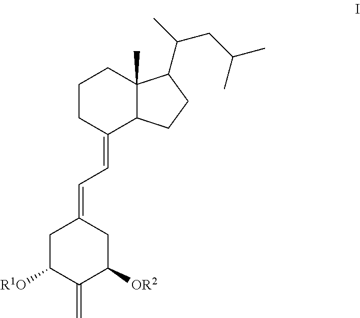

where R¹ and R² may be the same or different and are independently selected from H or hydroxy protecting groups.

In some embodiments, R¹ and R² are hydroxy protecting groups such as silyl groups. In some such embodiments, R¹ and R² are both t-butyldimethylsilyl groups. In other embodiments, R¹ and R² are both H such that the compound has the Formula II:

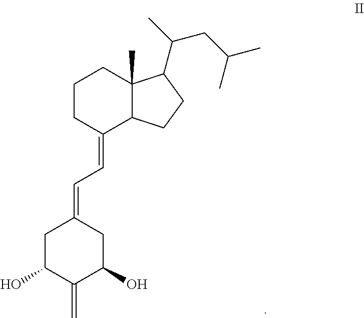 [SMILES: C=C1[C@H](O)CC(=C/C=C2\CCC[C@@]3(C)C2CCC3C(C)CC(C)C)C[C@H]1O]

In some embodiments, the carbon at position 20 of Formula II has the S configuration such that the compound has the Formula IIA and in other embodiments, the carbon at position 20 of the compound Formula II has an R configuration such that the compound has the Formula IIB:

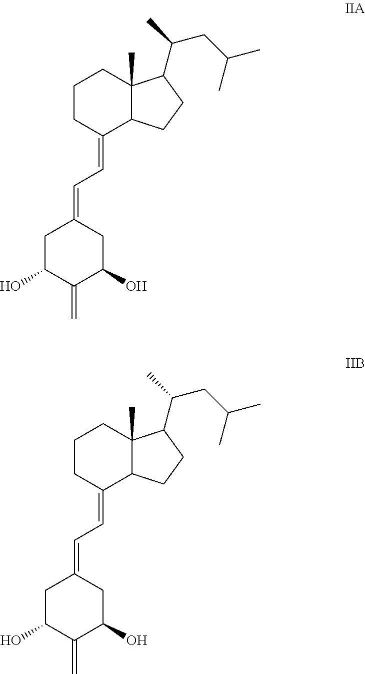 [SMILES: C=C1[C@H](O)CC(=C/C=C2\CCC[C@@]3(C)C2CCC3[C@@H](C)CC(C)C)C[C@H]1O ; C=C1[C@H](O)CC(=C/C=C2\CCC[C@@]3(C)C2CCC3[C@H](C)CC(C)C)C[C@H]1O]

In some embodiments, the carbon at position 17 of Formula IIA or IIB has the R configuration such that the compound has the Formula IIIA or IIIB, respectively:

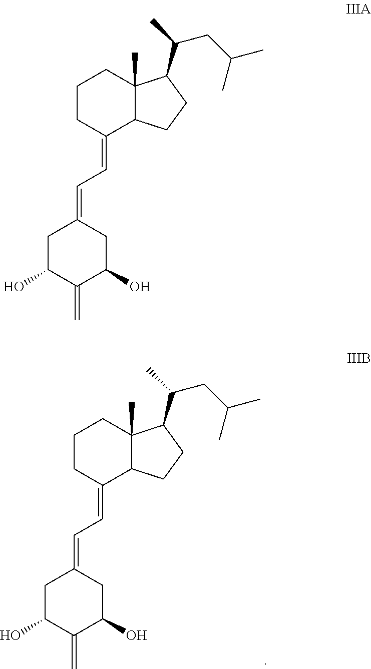 [SMILES: C=C1[C@H](O)CC(=C/C=C2\CCC[C@@]3(C)C2CC[C@@H]3[C@@H](C)CC(C)C)C[C@H]1O ; C=C1[C@H](O)CC(=C/C=C2\CCC[C@@]3(C)C2CC[C@@H]3[C@H](C)CC(C)C)C[C@H]1O]

Compounds of Formula I, II, IIA, IIB, IIIA, and IIIB show a highly advantageous pattern of biological activity, including binding to the vitamin D receptor (VDR), induction of 24-hydroxylase and HL-60 cell differentiation activities, and increasing intestinal calcium transport. Thus, the present compounds may be used in methods of preventing or treating certain biological conditions. The methods include administering an effective amount of a compound or a pharmaceutical composition comprising an effective amount of the compound to a subject, wherein the biological condition is selected from psoriasis; leukemia; colon cancer; breast cancer; prostate cancer; multiple sclerosis; lupus; diabetes mellitus; host versus graft reaction; rejection of organ transplants; an inflammatory disease selected from rheumatoid arthritis, asthma, or inflammatory bowel diseases; a skin condition selected from wrinkles, lack of adequate skin firmness, lack of adequate dermal hydration, or insufficient sebum secretion; or renal osteodystrophy.

A compound of the present technology may be present in a pharmaceutical composition to treat or prevent the above-noted diseases and disorders in an effective amount, optionally including a pharmaceutically acceptable carrier. In some embodiments, the amount of compound includes from about 0.01 μg per gram of the composition to about 1 mg per gram of the composition, alternatively from about 0.1 μg per gram to about 500 μg per gram of the composition, and may be administered topically, transdermally, orally, or parenterally in dosages of from about 0.01 μg per day to about 1 mg per day, or from about 0.1 μg per day to about 500 μg per day. In other embodiments, the compound or the composition may be administered by delivering the compound or the composition in aerosol.

Further features and advantages of the present technology will be apparent from the following detailed description and drawings.

### BRIEF DESCRIPTION OF THE DRAWINGS

FIGS. 1-5 illustrate various biological activities of (20S)-2-methylene-19,23,24-trinor-1α-hydroxyvitamin D₃ (referred to as “SMB” in the figures), compared with those of the native hormone, 1α,25-dihydroxyvitamin D₃ (referred to as “1,25(OH)₂D₃” in the figures). FIGS. 6-10 illustrate various biological activities of (20R)-2-methylene-19,23,24-trinor-1α-hydroxyvitamin D₃ (referred to as “RMB” in the figures) compared with those of the native hormone.

FIG. 1 is a graph showing the competitive binding of SMB and the native hormone, 1,25(OH)₂D₃ to the nuclear vitamin D hormone receptor.

FIG. 2 is a graph comparing the percent HL-60 cell differentiation as a function of the concentration of SMB with that of 1,25(OH)₂D₃.

FIG. 3 is a graph comparing the in vitro transcription activity of SMB with that of 1,25(OH)₂D₃.

FIG. 4 is a bar graph comparing the bone calcium mobilization activity of SMB with that of 1,25(OH)₂D₃ in rat.

FIG. 5 is a bar graph comparing the intestinal calcium transport activity of SMB with that of 1,25(OH)₂D₃ in rat.

FIG. 6 is a graph showing the competitive binding of RMB and 1,25(OH)₂D₃ to the nuclear vitamin D hormone receptor.

FIG. 7 is a graph comparing the percent HL-60 cell differentiation as a function of the concentration of RMB with that of 1,25(OH)₂D₃.

FIG. 8 is a graph comparing the in vitro transcription activity of RMB with that of 1,25(OH)₂D₃.

FIG. 9 is a bar graph comparing the bone calcium mobilization activity of RMB with that of 1,25(OH)₂D₃ in rat.

FIG. 10 is a bar graph comparing the intestinal calcium transport activity of RMB with that of 1,25(OH)₂D₃ in rat.

### DETAILED DESCRIPTION

(20S)-2-Methylene-19,23,24-trinor-1α-hydroxyvitamin D₃ and (20R)-2-methylene-19,23,24-trinor-1α-hydroxyvitamin D₃ were synthesized, tested, and found to be useful in treating or preventing a variety of biological conditions as described herein. Structurally, these compounds have the Formulas IIA (“SMB”) and IIB (“RMB”), as shown below:

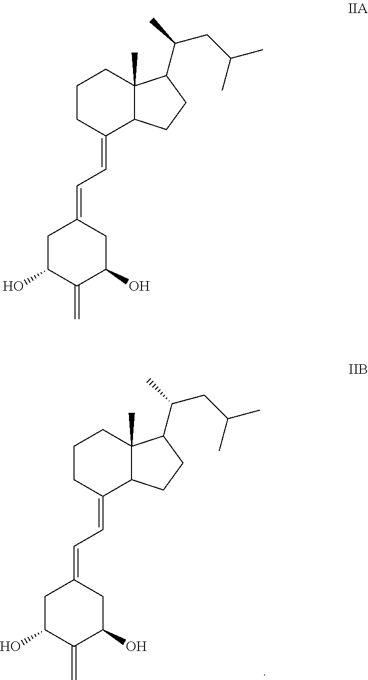 [SMILES: C=C1[C@H](O)CC(=C/C=C2\CCC[C@@]3(C)C2CCC3[C@@H](C)CC(C)C)C[C@H]1O ; C=C1[C@H](O)CC(=C/C=C2\CCC[C@@]3(C)C2CCC3[C@H](C)CC(C)C)C[C@H]1O]

In some embodiments, the compound of Formula IIA is a compound of Formula IIIA. In some embodiments, the compound of Formula IIB is a compound of Formula IIIB. The structures of the compounds of Formula IIIA and IIIB are shown below:

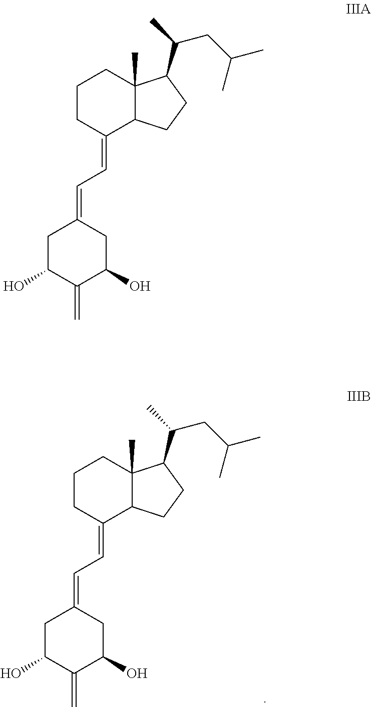 [SMILES: C=C1[C@H](O)CC(=C/C=C2\CCC[C@@]3(C)C2CC[C@@H]3[C@@H](C)CC(C)C)C[C@H]1O ; C=C1[C@H](O)CC(=C/C=C2\CCC[C@@]3(C)C2CC[C@@H]3[C@H](C)CC(C)C)C[C@H]1O]

As shown in Scheme 1, preparation of (20S)-2-methylene-19,23,24-trinor-1α-hydroxyvitamin D₃ and (20R)-2-methylene-19,23,24-trinor-1α-hydroxyvitamin D₃ can be generally accomplished starting from a single compound, aldehyde A. Aldehyde A is may be prepared from vitamin D₂ according to the methods disclosed by Fall et al., Tetrahedron Lett. 43, 1433 (2002), which is hereby incorporated by reference in its entirety and for all purposes as if fully set forth herein. Details for preparing the 20R- or the 20S-diastereomer of Windaus-Grundmann-type ketone B from aldehyde A are set forth in the Examples herein. Condensation of the appropriate ketone B with the allylic phosphine oxide reagent C, followed by deprotection of compound D (removal of the Y¹ and Y² groups) provides either diastereomer of 2-methylene-19,23,24-trinor-1α-hydroxyvitamin D₃ (E).

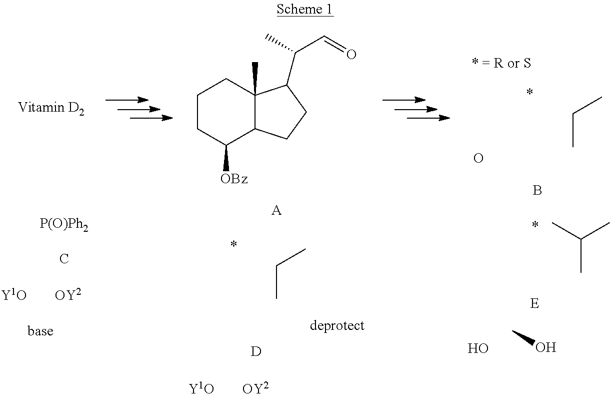

Specific examples of some other important Windaus-Grundmann-type ketones used to synthesize vitamin D analogs are those described in Mincione et al., Synth. Commun. 19, 723, (1989); and Peterson et al., J. Org. Chem. 51, 1948, (1986). An overall process for synthesizing 2-alkylidene-19-norvitamin D compounds is illustrated and described in U.S. Pat. No. 5,843,928, which is hereby incorporated by reference in its entirety and for all purposes as if fully set forth herein.

In phosphine oxide C, Y¹ and Y² are hydroxy protecting groups such as silyl protecting groups. The t-butyldimethylsilyl (i.e., TBDMS or TBS) group is an example of a particularly useful hydroxy protecting group. The process described above represents an application of the convergent synthesis concept, which has been applied effectively to the preparation of numerous vitamin D compounds (see Lythgoe et al., J. Chem. Soc. Perkin Trans. I, 590 (1978); Lythgoe, Chem. Soc. Rev. 9, 449 (1983); Toh et al., J. Org. Chem. 48, 1414 (1983); Baggiolini et al., J. Org. Chem. 51, 3098 (1986); Sardina et al., J. Org. Chem. 51, 1264 (1986); J. Org. Chem. 51, 1269 (1986); DeLuca et al., U.S. Pat. No. 5,086,191; DeLuca et al., U.S. Pat. No. 5,536,713; and DeLuca et al., U.S. Pat. No. 5,843,928, all of which are hereby incorporated by reference in their entirety and for all purposes as if fully set forth herein).

Phosphine oxide C is a convenient reagent that may be prepared according to the procedures described by Sicinski et al., J. Med. Chem. 41, 4662 (1998), DeLuca et al., U.S. Pat. No. 5,843,928; Perlman et al., Tetrahedron Lett. 32, 7663 (1991); and DeLuca et al., U.S. Pat. No. 5,086,191. Scheme 2 shows the general procedure for synthesizing phosphine oxide C (where Y¹ and Y² are TBDMS groups) as outlined in U.S. Pat. No. 5,843,928 which is hereby incorporated by reference in its entirety as if fully set forth herein.

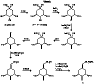

As used herein, the term “hydroxy protecting group” signifies any group used for the temporary protection of the hydroxy (—OH) functional group from unwanted chemical reactions. Non-limiting examples of types of hydroxy protecting groups include alkoxycarbonyl, acyl, alkoxyalkyl groups, and alkylsilyl or alkylarylsilyl groups (collectively referred to simply as “silyl” groups). Alkoxycarbonyl protecting groups are alkyl-O—CO-groups such as methoxycarbonyl, ethoxycarbonyl, propoxycarbonyl, isopropoxycarbonyl, butoxycarbonyl, isobutoxycarbonyl, tert-butoxycarbonyl, benzyloxycarbonyl or allyloxycarbonyl. The term “acyl” signifies an alkanoyl group of 1 to 6 carbons, in all of its isomeric forms, or a carboxyalkanoyl group of 1 to 6 carbons, such as an oxalyl, malonyl, succinyl, glutaryl group, or an aromatic acyl group such as benzoyl, or a halo, nitro or alkyl substituted benzoyl group. Alkoxyalkyl protecting groups are groups such as methoxymethyl, ethoxymethyl, methoxyethoxymethyl, or tetrahydrofuranyl and tetrahydropyranyl. Preferred silyl-protecting groups are trimethylsilyl, triethylsilyl, t-butyldimethylsilyl, dibutylmethylsilyl, diphenylmethylsilyl, phenyldimethylsilyl, diphenyl-t-butylsilyl and analogous alkylated silyl radicals. The term “aryl” specifies a phenyl-, or an alkyl-, nitro- or halo-substituted phenyl group. An extensive list of protecting groups for the hydroxy functionality may be found in Protective Groups in Organic Synthesis, Greene, T. W.; Wuts, P. G. M., John Wiley & Sons, New York, N.Y., (3rd Edition, 1999), which can be added or removed using the procedures set forth therein, and which is hereby incorporated by reference in its entirety and for all purposes as if fully set forth herein.

A “protected hydroxy” group is a hydroxy group derivatized or protected by any of the above groups commonly used for the temporary or permanent protection of hydroxy functional groups, e.g., the silyl, alkoxyalkyl, acyl or alkoxycarbonyl groups, as previously defined.

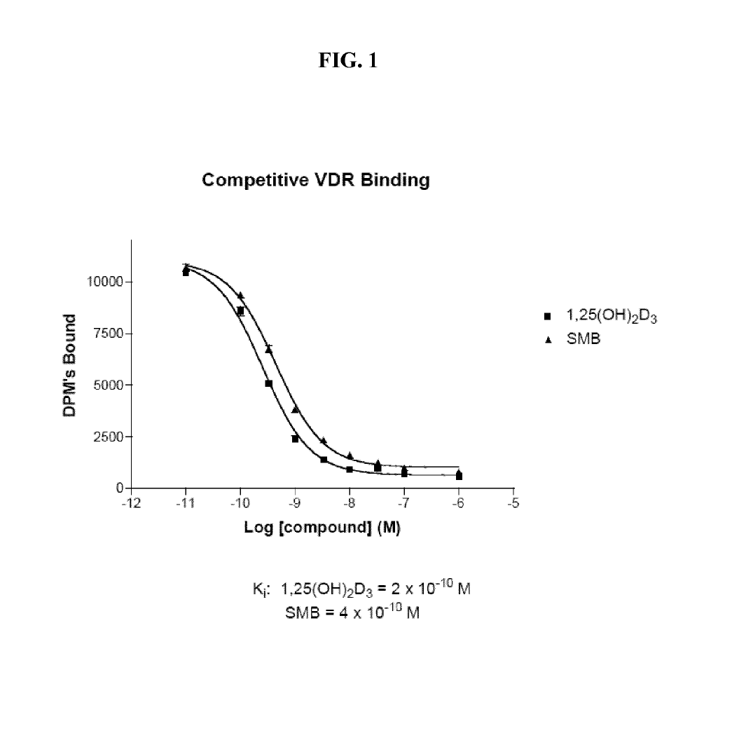

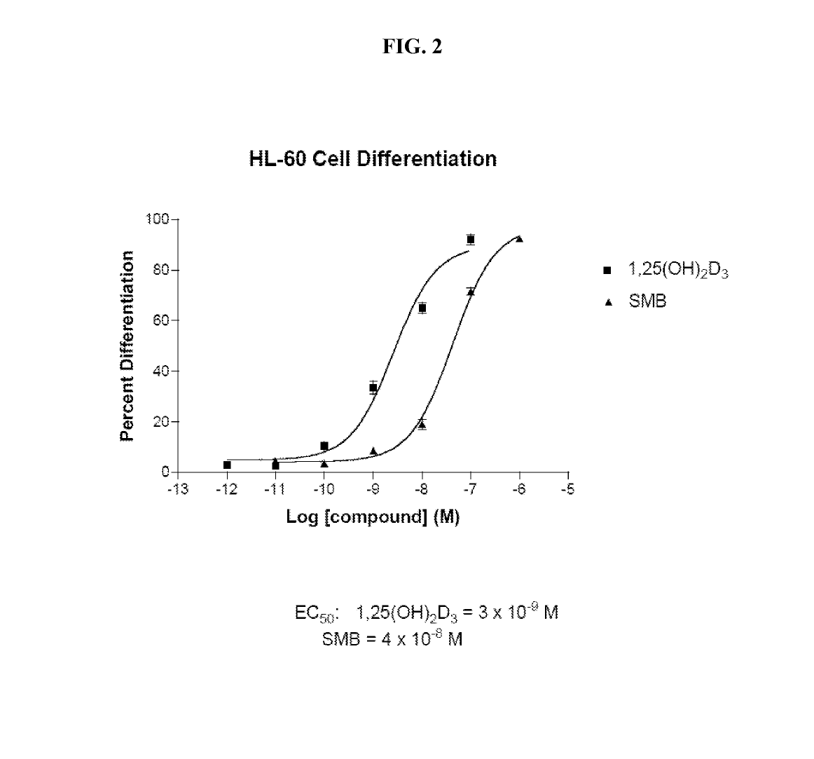

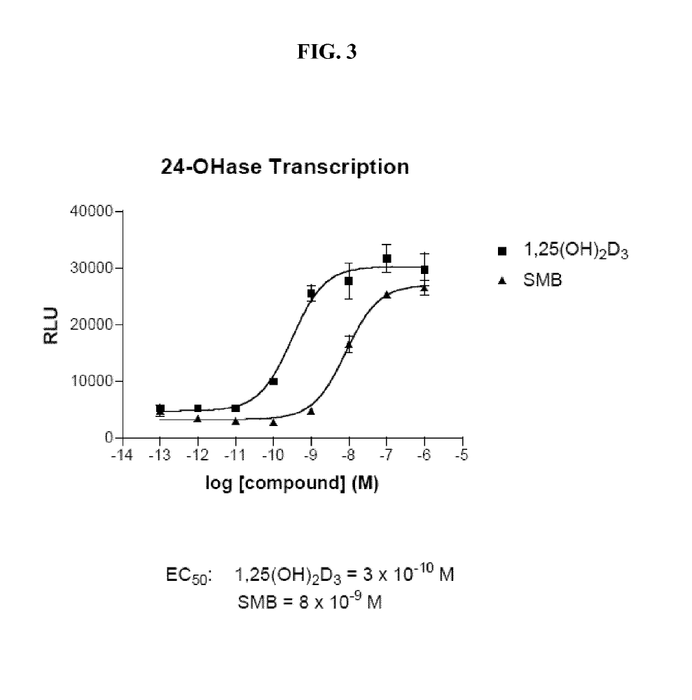

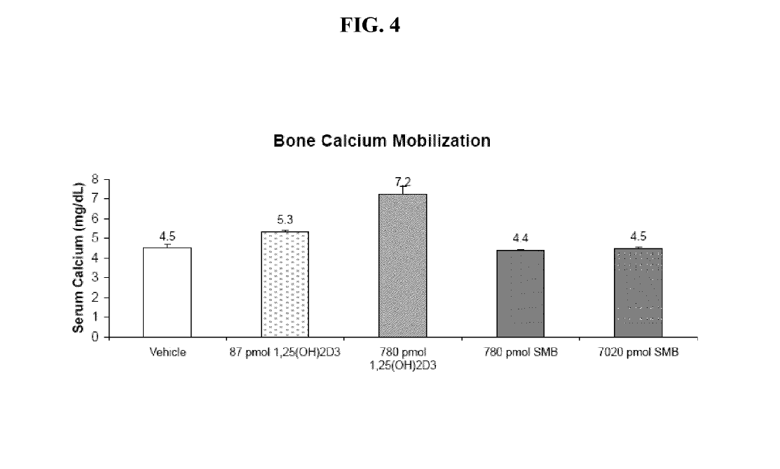

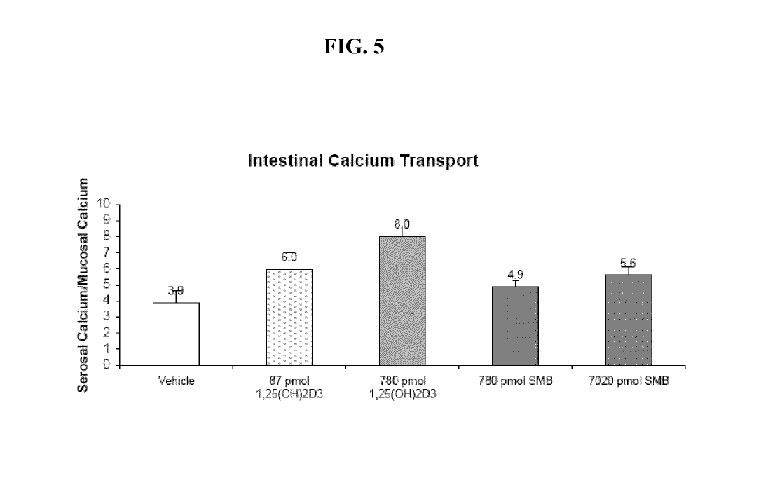

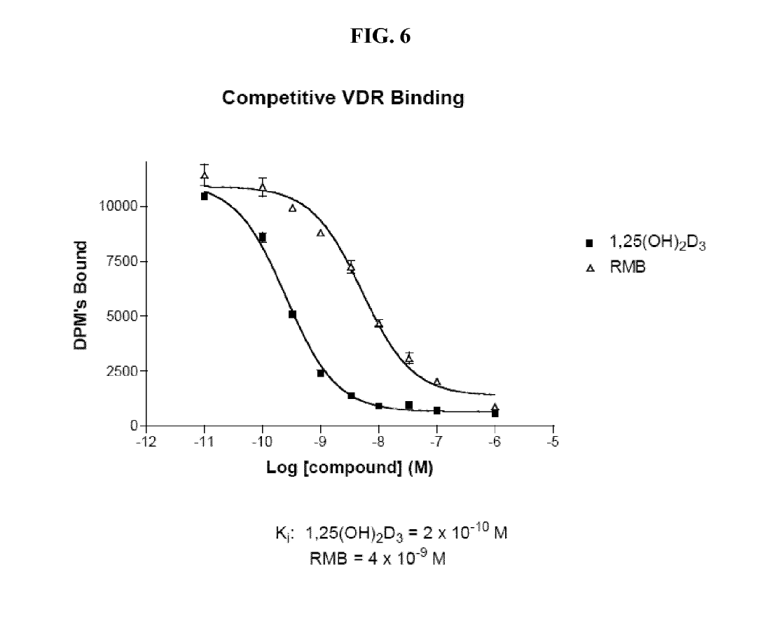

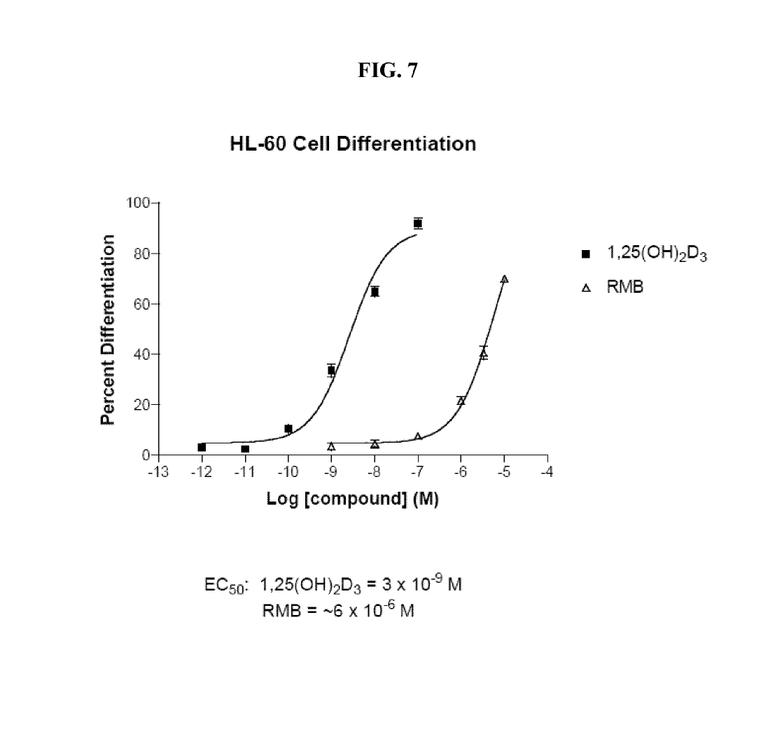

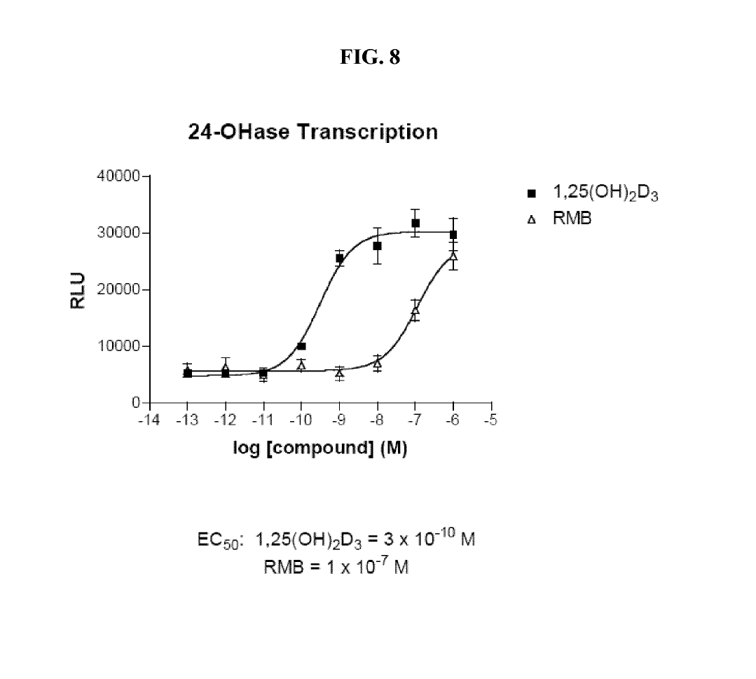

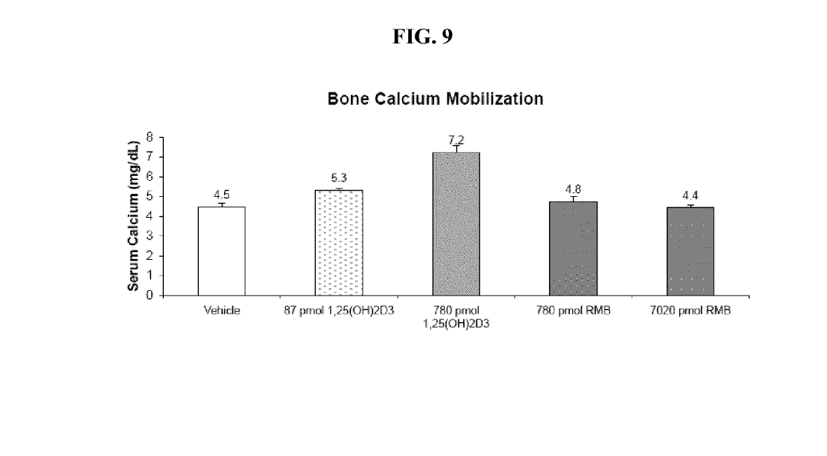

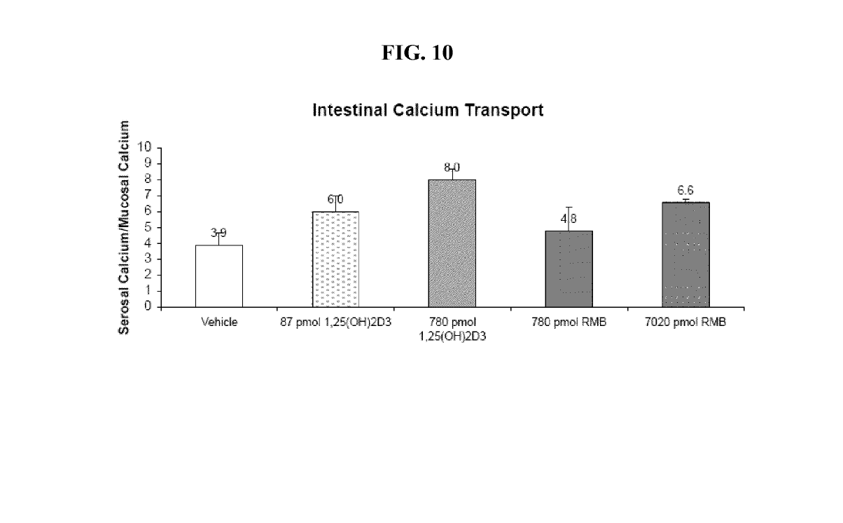

The compounds of the present technology show significant biological activity. SMB and RMB each strongly bind the vitamin D receptor, albeit with lower affinity relative to the native hormone, 1,25(OH)₂D₃ as shown in FIGS. 1 and 6. In comparison to the native hormone, SMB is less active than the native hormone in causing the differentiation of HL-60 cells into monocytes in culture (FIG. 2), but retains significant differentiation activity. Similarly, SMB is less active than the native hormone in increasing transcription of the 24-hydroxylase gene (FIG. 3), but retains significant activity. On the other hand, RMB shows more pronounced decreases in differentiation and transcription activities in comparison to the native hormone (FIGS. 7 and 8, respectively). Surprisingly, despite its reduced in vitro activity, RMB is active in vivo as evidenced by its intestinal calcemic activity in rats. As shown in FIG. 10, at doses of 7020 pmol (˜20 mcg/kg body weight), RMB elicits an elevation of the serosal/mucosal calcium higher than that of the native hormone at a dose of 87 pmol but less than the ratio of the native hormone at a dose of 780 pmol. The intestinal calcemic activity of SMB is similar to that of RMB (cf FIG. 5 and FIG. 10). As shown in FIGS. 4 and 9, neither SMB nor RMB show activity in the mobilization of bone calcium stores, even at high concentrations (e.g., 7020 pmol). Thus, the calcemic activities of SMB and RMB could be characterized as intestinal-specific.

In view of their biological activity, compounds of the present technology may be used for treatment and prophylaxis of human disorders which are characterized by an imbalance in the immune system, e.g., in autoimmune diseases, including multiple sclerosis, lupus, diabetes mellitus, host versus graft reaction, and rejection of organ transplants; and additionally for the treatment of inflammatory diseases, such as rheumatoid arthritis, asthma, and inflammatory bowel diseases such as celiac disease, ulcerative colitis and Crohn's disease. Acne, alopecia and hypertension are other conditions which may be treated with the compounds of the present technology. Furthermore, since the compounds of the present technology can be characterized as having low calcemic activity, they may be especially useful in treating or preventing diseases where elevation of calcium (hypercalcemia) is undesirable.

In view of the their cellular differentiation activity, the present compounds may also be used in the treatment of psoriasis, or as anti-cancer agents, especially against leukemia, colon cancer, breast cancer and prostate cancer. In addition, these compounds provide a therapeutic agent for the treatment of various skin conditions including wrinkles, lack of adequate dermal hydration, i.e., dry skin, lack of adequate skin firmness, i.e., slack skin, and insufficient sebum secretion. Use of these compounds thus not only results in moisturizing of skin but also improves the barrier function of skin.

The compounds of the present technology may be used to prepare pharmaceutical compositions, formulations, or medicaments that include a compound of the present technology in combination with a pharmaceutically acceptable carrier. Such pharmaceutical compositions, formulations, and medicaments may be used to treat or prevent various biological disorders such as those described herein. Methods for treating or preventing such disorders typically include administering an effective amount of the compound or an appropriate amount of a pharmaceutical composition, formulation, or a medicament that includes the compound to a subject suffering, or which may suffer, from the biological disorder. In some embodiments, the subject is a mammal. In some such embodiments, the mammal is selected from a rodent, a primate, a bovine, an equine, a canine, a feline, an ursine, a porcine, a rabbit, or a guinea pig. In some such embodiments, the mammal is a rat or is a mouse. In some embodiments, the subject is a primate such as, in some embodiments, a human.

For treatment purposes, the compounds defined by Formula I, II, IIA, IIB, IIIA, and IIIB may be formulated for pharmaceutical applications as a solution in innocuous solvents, or as an emulsion, suspension or dispersion in suitable solvents or carriers, or as pills, tablets or capsules, together with solid carriers, according to conventional methods known in the art. Any such formulations may also contain other pharmaceutically acceptable and non-toxic excipients such as stabilizers, anti-oxidants, binders, coloring agents or emulsifying or taste-modifying agents. Pharmaceutically acceptable excipients and carriers are generally known to those skilled in the art and are thus included in the present technology. Such excipients and carriers are described, for example, in “Remingtons Pharmaceutical Sciences,” Mack Pub. Co., New Jersey (1991), which is hereby incorporated by reference in its entirety and for all purposes as if fully set forth herein.

The compounds may be administered orally, topically, parenterally, or transdermally. The compounds are advantageously administered by injection or by intravenous infusion or suitable sterile solutions, or in the form of liquid or solid doses via the alimentary canal, or in the form of creams, ointments, patches, or similar vehicles suitable for transdermal applications. In some embodiments, doses of from 0.001 μg to about 1 mg per day of the compound are appropriate for treatment purposes. In some such embodiments, an appropriate and effective dose may range from 0.01 μg to 1 mg per day of the compound. In other such embodiments, an appropriate and effective dose may range from 0.1 μg to 500 μg per day of the compound. Such doses will be adjusted according to the type of disease or condition to be treated, the severity of the disease or condition, and the response of the subject as is well understood in the art. The compound may be suitably administered alone, or together with another active vitamin D compound.

Compositions for use in the present technology include an effective amount of the compound of Formula I, II, IIA, IIB, IIIA, or IIIB as the active ingredient, and a suitable carrier. An effective amount of the compound for use in accordance with some embodiments of the present technology will generally be a dosage amount such as those described herein, and may be administered topically, transdermally, orally, nasally, rectally, or parenterally.

The compound of Formula I, II, IIA, IIB, IIIA, or IIIB may be advantageously administered in amounts sufficient to effect the differentiation of promyelocytes to normal macrophages. Dosages as described above are suitable, it being understood that the amounts given are to be adjusted in accordance with the severity of the disease, and the condition and response of the subject as is well understood in the art.

The compound may be formulated as creams, lotions, ointments, aerosols, suppositories, topical patches, pills, capsules or tablets, or in liquid form as solutions, emulsions, dispersions, or suspensions in pharmaceutically innocuous and acceptable solvent or oils, and such preparations may contain, in addition, other pharmaceutically innocuous or beneficial components, such as stabilizers, antioxidants, emulsifiers, coloring agents, binders or taste-modifying agents.

The formulations of the present technology comprise an active ingredient in association with a pharmaceutically acceptable carrier and, optionally, other therapeutic ingredients. The carrier must be “acceptable” in the sense of being compatible with the other ingredients of the formulations and not deleterious to the recipient thereof.

Formulations of the present technology suitable for oral administration may be in the form of discrete units as capsules, sachets, tablets or lozenges, each containing a predetermined amount of the active ingredient; in the form of a powder or granules; in the form of a solution or a suspension in an aqueous liquid or non-aqueous liquid; or in the form of an oil-in-water emulsion or a water-in-oil emulsion.

Formulations for rectal administration may be in the form of a suppository incorporating the active ingredient and carrier such as cocoa butter, or in the form of an enema.

Formulations suitable for parenteral administration conveniently comprise a sterile oily or aqueous preparation of the active ingredient which is preferably isotonic with the blood of the recipient.

Formulations suitable for topical administration include liquid or semi-liquid preparations such as liniments, lotions, oil-in-water or water-in-oil emulsions such as creams, ointments or pastes; or solutions or suspensions such as drops; or as sprays.

For nasal administration, inhalation of powder, self-propelling or spray formulations, dispensed with a spray can, a nebulizer or an atomizer can be used. The formulations, when dispensed, preferably have a particle size in the range of 10 to 100 microns.

The formulations may conveniently be presented in dosage unit form and may be prepared by any of the methods well known in the art of pharmacy. By the term “dosage unit” is meant a unitary, i.e., a single dose which is capable of being administered to a patient as a physically and chemically stable unit dose comprising either the active ingredient as such or a mixture of it with solid or liquid pharmaceutical diluents or carriers.

All references cited herein are specifically incorporated by reference in their entirety and for all purposes as if fully set forth herein.

The present technology is further illustrated by the following examples, which should not be construed as limiting in any way.

### EXAMPLES

### Example 1

### Synthesis of (20S)- and (20R)-2-methylene-19,23,24-trinor-1α-hydroxyvitamin D₃

Compounds of Formula I, Formula II, Formula IIA, Formula IIB, Formula IIIA, and Formula IIIB were prepared using the methods shown in Scheme 3. As shown in Scheme 3, compound I was obtained from vitamin D₂ as described by Fall et al., Tetrahedron Lett. 43, 1433, (2002). Treatment of the aldehyde 1 with hydroxylamine hydrochloride followed by dehydration of the intermediate E- and Z-oxime mixture with acetic anhydride provided nitrile 2. Alkylation of the anion of nitrile 2 with isobutyl bromide provided compound 3. X-Ray analysis established that compound 3 possesses 20S configuration (referring to vitamin D₃ numbering, i.e., the nitrile-bearing carbon possesses and S-configuration). Alkaline hydrolysis of the 8β-benzoyloxy group with methanolic potassium hydroxide provided alcohol 4. Reductive decyanation of alcohol 4 with potassium metal in HMPA gave a 1:1 mixture of epimeric alcohols, 5a and 5b. The reductive decyanation may also be performed using potassium metal and dicyclohexano-18-crown-6 in toluene. While protection of the 8β-hydroxy group of alcohol 4 may be performed prior to reductive decyanation (e.g., as an alkylsilyl-, arylsilyl-, or alkoxyalkyl ether), it was found that such protection was not necessary. Oxidation of the mixture of C-20 epimeric alcohols with tetrapropylammonium perruthenate followed by HPLC separation, provided separated hydrindanones 6a and 6b. Wittig-Horner condensation of 6a with known phosphine oxide 7 in the presence of phenyllithium provided bis(silyl ether) 8a. Deprotection of the silyl ethers with TBAF in THF provided compound 9a (RMB). Similarly, Wittig-Horner condensation of 6b with phosphine oxide 7 gave bis(silyl ether) 8b which was deprotected to provide compound 9b (SMB).

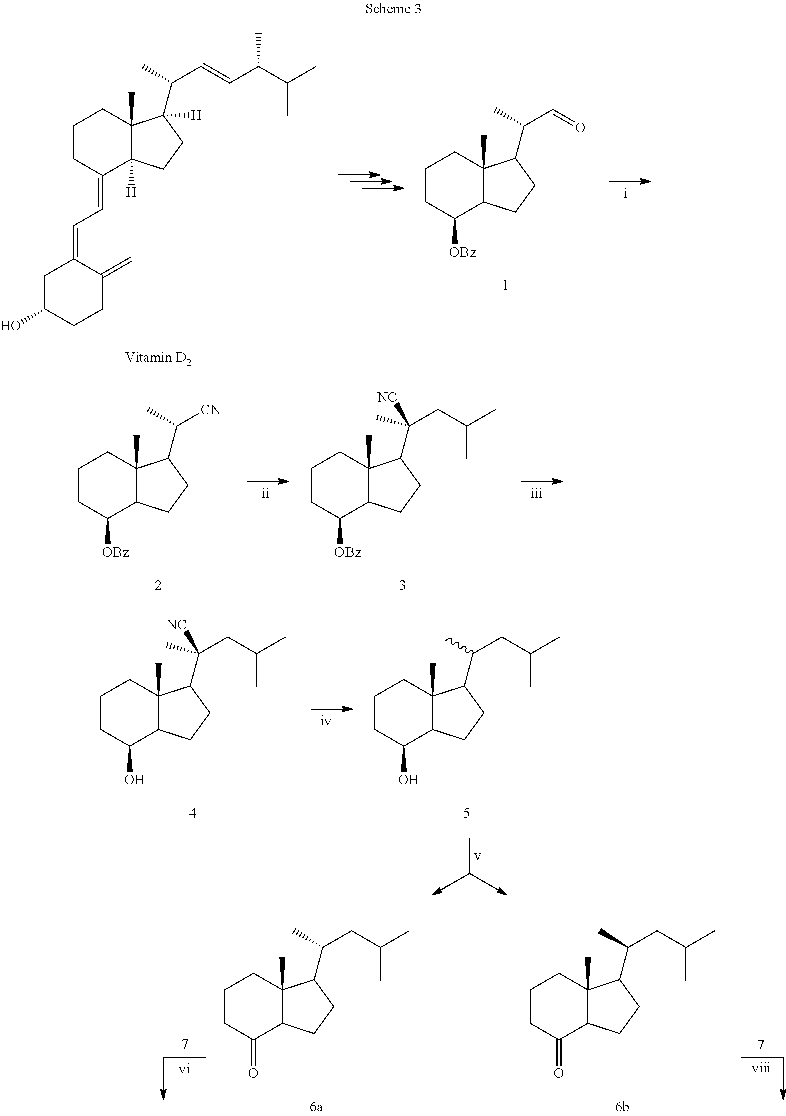

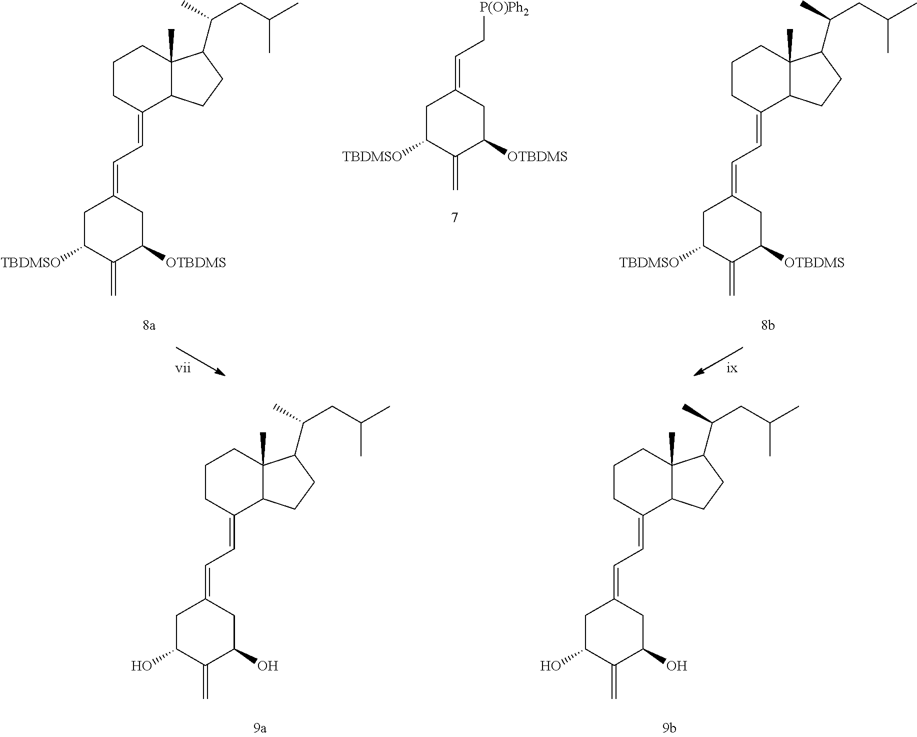

(i) NH₂OH.HCl, py, 89%, then Ac₂O, 91%; (ii) LDA, (CH₃)₂CHCH₂Br, THF, 66% (74% bsm); (iii) KOH, MeOH, 92%; (iv) K metal, Et₂O, t-BuOH, HMPA, 84%; (v) TPAP, NMO, CH₂Cl₂, then HPLC separation, 91% (6a+6b); (vi) 7, PhLi, THF, 50%; TBAF, THF, 81%; (viii) 7, PhLi, THF, 9%; (ix) TBAF, THF, 86%.

### (1R,3aR,4S,7aR)-1-((S)-1′-Cyanoethyl)-7a-methyloctahydroinden-4-yl benzoate (2)

To a solution of a benzoyloxy aldehyde 1 (284 mg, 0.90 mmol) in anhydrous pyridine (5 mL) was added NH₂OH.HCl (210 mg) and the mixture was stirred at room temperature for 20 hours. Then it was poured into water and extracted with ethyl acetate. The combined organic phases were separated, washed with saturated NaHCO₃ solution, water, and saturated CuSO₄ solution, dried (MgSO₄), and evaporated. The oily residue was purified by column chromatography on silica gel. Elution with hexane/ethyl acetate (9:1) gave pure, less polar E-oxime (167 mg) and more polar Z-oxime (105 mg, total yield 89%).

E-oxime: ¹H NMR (400 MHz, CDCl₃) δ 1.09 (3H, d, J=6.7 Hz, 18-H₃), 1.14 (3H, s, 21-H₃), 2.40 (1H, m, 20-H), 5.42 (1H, narr m, 8α-H), 7.27 (1H, d, J=8.0 Hz, 22-H), 7.45 (2H, t, J˜7 Hz, Ar—H), 7.56 (1H, t, J=7.4 Hz, Ar—H), 8.04 (2H, d, J=7.4 Hz, Ar—H).

Z-oxime: ¹H NMR (400 MHz, CDCl₃) δ 1.09 (3H, d, J=6.7 Hz, 18-H₃), 1.13 (3H, s, 21-H₃), 3.28 (1H, m, 20-H), 5.42 (1H, narr m, 8α-H), 6.25 (1H, d, J=8.1 Hz, 22-H), 7.45 (2H, t, J˜7 Hz, Ar—H), 7.56 (1H, t, J=7.3 Hz, Ar—H), 8.04 (2H, d, J=7.3 Hz, Ar—H).

The solution of the oximes (both isomers, 248 mg, 0.75 mmol) in acetic anhydride (8 mL) was refluxed for 1.5 hours. The reaction mixture was cooled, poured carefully into saturated solution of NaHCO₃ in water and extracted with toluene. Extracts were combined, washed with water, NaHCO₃ and brine, dried (MgSO₄) and evaporated. The residue was applied on a silica Sep-Pak (5 g). Elution with hexane/ethyl acetate (95:5) gave pure semi-crystalline nitrile 2 (212 mg, 91%). 2: [α]²⁴_{D} +81.5 (c 0.9, CHCl₃); ¹H NMR (400 MHz, CDCl₃) δ 1.124 (3H, s, 7a-CH₃), 1.373 (3H, d, J=7.1 Hz, 2′-H₃), 1.90 (1H, br d, J=12.8 Hz, 5β-H), 2.68 (1H, pentet, J=7.0 Hz, 1′-H), 5.43 (1H, narr m, 4α-H), 7.45 (2H, t, J=7.6 Hz, Ar—H), 7.57 (1H, t, J=7.5 Hz, Ar—H), 8.03 (2H, d, J=7.4 Hz, Ar—H); HRMS (ESI) exact mass calcd for C₁₃H₂₀ON (M⁺−C₆H₅CO) 206.1545, measured 206.1539.

### (1S,3aR,4S,7aR)-1-((S)-2′-Cyano-4′-methylpentan-2′-yl)-7a-methyloctahydroinden-4-yl benzoate (3)

n-Butyllithium (2.65 M in hexanes, 103 μL, 0.272 mmol) was added at 0° C. to the flask containing diisopropylamine (42 μL, 0.272 mmol) and THF (0.4 mL). The solution was stirred at 0° C. for 20 minutes, cooled to −78° C. and siphoned to the solution of 2 (77 mg, 0.248 mmol) in THF (0.3 mL). The resulted yellow mixture was stirred for 30 min, then HMPA (100 μL) was added and stirring was continued for another 15 minutes. Then, (CH₃)₂CHCH₂Br (68 μL, 0.62 mmol) was added, the solution was allowed to warm up to −40° C. during 1 hour, and saturated NH₄Cl was added. The mixture was extracted with ethyl acetate, the combined organic phases were washed with water, dried (MgSO₄) and evaporated. The residue was applied on a silica Sep-Pak (2 g). Elution with hexane/ethyl acetate (98:2) resulted in pure semi-crystalline 3 (60 mg, 66%; 74% based on recovered substrate); further elution with hexane/ethyl acetate (97:3) gave unreacted 2 (8.5 mg). 3: [α]²⁴_{D} +66.5 (c 1.15, CHCl₃); ¹H NMR (400 MHz, CDCl₃) δ 1.055 and 0.971 (3H and 3H, each d, J=6.6 Hz, 4′-CH₃ and 5′-H₃), 1.369 (3H, s, 7a-CH₃), 1.456 (3H, s, 1′-H₃), 2.15 (1H, br d, J=12.7 Hz, 5β-H), 5.40 (1H, narr m, 4α-H), 7.45 (2H, t, J˜7 Hz, Ar—H), 7.57 (1H, t, J=7.4 Hz, Ar—H), 8.04 (2H, d, J=7.4 Hz, Ar—H); HRMS (ESI) exact mass calcd for C₂₄H₃₃O₂N (M+) 367.2511, measured 367.2518.

### (S)-2-((1′S,3a′R,4′S,7a′R)-4′-hydroxy-7a′-methyloctahydroinden-1′-yl)-2,4-dimethylpentanenitrile (4)

Benzoyloxy nitrile 3 (90 mg, 0.246 mmol) was treated with 10% methanolic KOH (4 mL) at 50° C. for 18 hours. After concentration under vacuum the reaction mixture was poured into water and extracted with benzene and ether. The organic extracts were combined, washed with brine, dried (MgSO₄) and evaporated. The residue was redissolved in hexane/ethyl acetate (95:5) and the solution passed through a silica gel Sep-Pak cartridge. Evaporation of solvents gave hydroxy nitrile 4 (66 mg, 92%). 4: [α]²⁴_{D} +28 (c 0.29, CHCl₃); ¹H NMR (400 MHz, CDCl₃) δ 1.043 and 0.959 (3H and 3H, 2×d, J=6.6 Hz, 4-CH₃ and 5-H₃), 1.236 (3H, s, 7a′-CH₃), 1.410 (3H, s, 2-CH₃), 2.08 (1H, dm, J=12.4 Hz, 5′β-H), 4.09 (1H, narr m, 4′α-H), HRMS (ESI) exact mass calcd for C₁₇H₂₉ON (M+) 263.2249, measured 263.2254.

### (1R,3aR,4S,7aR)-7a-Methyl-1-((R)-4′-methylpentan-2′-yl)octahydroinden-4-ol (5a) and (1R,3aR,4S,7aR)-7a-Methyl-1-((S)-4′-methylpentan-2′-yl)octahydroinden-4-ol (5b)

A solution of nitrile 4 (49 mg, 0.186 mmol) in t-BuOH (50 μL) and ether (0.20 mL) was added drop-wise at 0° C., under argon, to the blue solution of potassium (55 mg, 1.4 mmol) in HMPA (0.17 mL) and ether (0.42 mL). The cooling bath was removed and the stirring was continued for 4 hours at room temperature under argon. The reaction was diluted with benzene, the unreacted potassium was removed and few drops of 2-propanol were added. The organic phase was washed with water, dried (MgSO₄) and evaporated. The residue was applied on a silica Sep-Pak (2 g). Elution with hexane/ethyl acetate (95:5) gave 1:1 mixture of epimeric alcohols 5a and 5b (37 mg, 84%). 5a and 5b: ¹H NMR (400 MHz, CDCl₃, selected signals) δ 0.932 (s, 7a-CH₃ in 5b), 0.944 (s, 7a-CH₃ in 5a), 2.01 (br d, J=12.7 Hz, 5β-H from both isomers), 4.07 (narr m, 4α-H from both isomers); HRMS (ESI) exact mass calcd for C₁₆H₃₀O (M+) 238.2297, measured 238.2294.

### (1R,3aR,7aR)-7a-methyl-1-((R)-4′-methylpentan-2′-yl)hexahydroinden-4(2H)-one (6a) and (1R,3aR,7aR)-7a-methyl-1-((S)-4′-methylpentan-2′-yl)hexahydroinden-4(2H)-one

The solution of NMO (23 mg) and molecular sieves 4 Å (123 mg) in methylene chloride (0.9 mL) was stirred at room temperature for 15 minutes, then the solution of 5a and 5b (20.5 mg, 86 μmol) in methylene chloride (0.15 mL) was added followed by TPAP (2.5 mg). The resultant dark mixture was stirred for 30 minutes, diluted with methylene chloride and filtered through a silica Sep-Pak (2 g). Elution with methylene chloride gave a 1:1 mixture of epimeric ketones 6a and 6b (21 mg, 91%). The separation of isomers was achieved by HPLC (9.4 mm×25 cm Zorbax-Sil column, 4 mL/min) using hexane/ethyl acetate (95:5) solvent system. The 20S-ketone 6b was collected at R_{V} 39 mL and the 20R-isomer 6a at R_{V} 40 mL. 6a: [α]²⁴_{D} +11 (c 0.28, CHCl₃); ¹H NMR (400 MHz, CDCl₃) δ 0.653 (3H, s, 7a-CH₃), 0.816 and 0.881 (3H and 3H, each d, J=6.6 Hz, 4′-CH₃ and 5-H₃), 0.922 (3H, d, J=5.9 Hz, 1′-H₃), 2.14 (1H, br d, J=12.4 Hz, 5β-H), 2.44 (1H, dd, J=11.6, 7.6 Hz, 3aα-H); HRMS (ESI) exact mass calcd for C₁₆H₂₈O (M+) 236.2140, measured 236.2135. 6b: [α]²⁴_{D} −48 (c 0.28, CHCl₃); ¹H NMR (400 MHz, CDCl₃) δ 0.641 (3H, s, 7a-CH₃), 0.827 and 0.831 (3H and 3H, each d, J=6.6 Hz, 4′-CH₃ and 5′-H₃), 0.894 (3H, d, J=5.9 Hz, 1′-H₃), 2.12 (1H, br d, J=12.7 Hz, 5β-H), 2.44 (1H, dd, J=11.5, 7.6 Hz, 3a4α-H); HRMS (ESI) exact mass calcd for C₁₆H₂₈O (M+) 236.2140, measured 236.2135.

### 1α-[(tert-Butyldimethylsilyl)oxy]-2-methylene-19,23,24-trinorvitamin D₃ tert-butyldimethylsilyl ether (8a) and (20R)-2-methylene-19,23,24-trinor-1α-hydroxyvitamin

To a solution of phosphine oxide 7 (24 mg, 42 μmol) in anhydrous THF (0.6 mL) at −78° C. was slowly added phenyllithium (1.8 M in butyl ether, 24 μL, 42 μmol) under argon with stirring. The solution turned deep orange. The mixture was stirred at −78° C. for 20 minutes and a precooled (−78° C.) solution of the ketone 6a (5.5 mg, 23 μmol) in anhydrous THF (0.1 mL) was slowly added. The mixture was stirred under argon at −78° C. for 2 hours and at 6° C. for 16 hours. Ethyl acetate and water were added, and the organic phase was washed with brine, dried (MgSO₄), and evaporated. The residue was dissolved in hexane, applied on a silica Sep-Pak cartridge, and eluted with hexane/ethyl acetate (95.5:0.5) to give 19-norvitamin derivative 8a (7.0 mg, 50%). The Sep-Pak was then washed with hexane/ethyl acetate (98:2) to recover some unchanged C,D-ring ketone 6a (1 mg).

To a solution of the protected vitamin 8a (7 mg, 11.6 μmol) in anhydrous THF (7.5 mL) was added tetrabutylammonium fluoride (1.0 M in THF, 348 μL, 348 μmol) and triethylamine (65 μL). The mixture was stirred under argon at room temperature for 18 hours, poured into brine and extracted with ethyl acetate and diethyl ether. Organic extracts were washed with brine, dried (MgSO₄), and evaporated. The residue was purified by HPLC (9.4 mm×25 cm Zorbax-Sil column, 4 mL/min) using hexane/2-propanol (9:1) solvent system. Pure 19-norvitamin 9a (3.5 mg, 81%) was collected at R_{V} 24 mL. In reversed-phase HPLC (9.4 mm×25 cm Eclipse XDB-C18 column, 4 mL/min) using methanol/water (95:5) solvent system, vitamin 9 was collected at R_{V} 43 mL. 9a: UV (in EtOH) λₘₐₓ 244.5, 252.0, 261.5; ¹H NMR (400 MHz, CDCl₃) δ 0.566 (3H, s, 18-H₃), 0.820 and 0 879 (3H and 3H, each d, J=6.5 Hz, 26- and 27-H₃), 0.913 (3H, d, J=6.5 Hz, 21-H₃), 2.29 (1H, dd, J=13.3, 8.5 Hz, 10α-H), 2.33 (1H, dd, J=13.3, 6.1 Hz, 4β-H), 2.58 (1H, dd, J=13.3, 3.7 Hz, 4α-H), 2.83 (2H, m, 9β-H and 10β-H), 4.48 (2H, m, 1β- and 3α-H), 5.09 and 5.11 (1H and 1H, each s, H₂C═), 5.88 and 6.36 (1H and 1H, each d, J=11.2 Hz, 7- and 6-H).

### (20S)-1α-[(tert-Butyldimethylsilyl)oxy]-2-methylene-19,23,24-trinorvitamin D₃ tert-butyldimethylsilyl ether (8b) and (20S)-2-methylene-19,23,24-trinor-1α-hydroxyvitamin D₃ (9b)

To a solution of phosphine oxide 7 (24 mg, 42 μmol) in anhydrous THF (0.6 mL) at −78° C. was slowly added phenyllithium (1.8 M in butyl ether, 24 μL, 42 μmol) under argon with stirring. The solution turned deep orange. The mixture was stirred at −78° C. for 20 minutes and a precooled (−78° C.) solution of the ketone 6b (5.5 mg, 23 μmol) in anhydrous THF (0.1 mL) was slowly added. The mixture was stirred under argon at −78° C. for 2 hours and at 6° C. for 16 hours. Ethyl acetate and water were added, and the organic phase was washed with brine, dried (MgSO₄), and evaporated. The residue was dissolved in hexane, applied on a silica Sep-Pak cartridge, and eluted with hexane/ethyl acetate (95.5:0.5) to give 19-norvitamin derivative 8b (1.2 mg, 9%). The Sep-Pak was then washed with hexane/ethyl acetate (98:2) to recover some unchanged C,D-ring ketone 6b (2 mg), and with hexane/ethyl acetate (6:4) to recover diphenylphosphine oxide 7 (4 mg).

To a solution of the protected vitamin 8b (1.2 mg, 2 μmol) in anhydrous THF (1.3 mL) was added tetrabutylammonium fluoride (1.0 M in THF, 60 μL, 60 mol) and triethylamine (11 μL). The mixture was stirred under argon at room temperature for 18 hours, poured into brine and extracted with ethyl acetate and diethyl ether. Organic extracts were washed with brine, dried (MgSO₄), and evaporated. The residue was purified by HPLC (9.4 mm×25 cm Zorbax-Sil column, 4 mL/min) using hexane/2-propanol (9:1) solvent system. Pure 19-norvitamin 9b (0.64 mg, 86%) was collected at R_{V} 24 mL. In reversed-phase HPLC (9.4 mm×25 cm Eclipse XDB-C18 column, 4 mL/min) using methanol/water (95:5) solvent system, vitamin 9b was collected at R_{V} 41 mL. 9b: UV (in EtOH) λₘₐₓ 244.5, 252.0, 261.5 nm; ¹H NMR (400 MHz, CDCl₃) δ 0.549 (3H, s, 18-H₃), 0.879 (3H, d, J=6.5 Hz, 21-H₃), 0.815 and 0.824 (3H and 3H, each d, J=6.5 Hz, 26- and 27-H₃), 2.30 (1H, dd, J=13.3, 8.5 Hz, 10α-H), 2.33 (1H, dd, J=13.3, 6.1 Hz, 4β-H), 2.58 (1H, dd, J=13.3, 3.7 Hz, 4α-H), 2.83 (2H, m, 9β- and 10β-H), 4.48 (2H, m, 1β- and 3α-H), 5.09 and 5.11 (1H and 1H, each s, H₂C═), 5.89 and 6.36 (1H and 1H, each d, J=11.2 Hz, 7- and 6-H).

### Example 2

### Biological Activity

### Vitamin D Receptor Binding

### Test Material

### Protein Source

Full-length recombinant rat receptor was expressed in E. coli BL21(DE3) Codon Plus RIL cells and purified to homogeneity using two different column chromatography systems. The first system was a nickel affinity resin that utilizes the C-terminal histidine tag on this protein. The protein that was eluted from this resin was further purified using ion exchange chromatography (S-Sepharose Fast Flow). Aliquots of the purified protein were quick frozen in liquid nitrogen and stored at −80° C. until use. For use in binding assays, the protein was diluted in TEDK₅₀ (50 mM Tris, 1.5 mM EDTA, pH 7.4, 5 mM DTT, 150 mM KCl) with 0.1% Chaps detergent. The receptor protein and ligand concentration was optimized such that no more than 20% of the added radiolabeled ligand was bound to the receptor.

### Study Drugs

Unlabeled ligands were dissolved in ethanol and the concentrations determined using UV spectrophotometry (1,25(OH)₂D₃: molar extinction coefficient=18,200 and λₘₐₓ=265 nm; Analogs: molar extinction coefficient=42,000 and λmax=252 nm). Radiolabeled ligand (³H-1,25(OH)₂D₃, ˜159 Ci/mmole) was added in ethanol at a final concentration of 1 nM.

### Assay Conditions

Radiolabeled and unlabeled ligands were added to 100 mcl of the diluted protein at a final ethanol concentration of ≦10%, mixed and incubated overnight on ice to reach binding equilibrium. The following day, 100 mcl of hydroxylapatite slurry (50%) was added to each tube and mixed at 10-minute intervals for 30 minutes. The hydroxylapatite was collected by centrifugation and then washed three times with Tris-EDTA buffer (50 mM Tris, 1.5 mM EDTA, pH 7.4) containing 0.5% Titron X-100. After the final wash, the pellets were transferred to scintillation vials containing 4 ml of Biosafe II scintillation cocktail, mixed and placed in a scintillation counter. Total binding was determined from the tubes containing only radiolabeled ligand.

### HL-60 Differentiation

### Test Material

### Study Drugs

The study drugs were dissolved in ethanol and the concentrations determined using UV spectrophotometry. Serial dilutions were prepared so that a range of drug concentrations could be tested without changing the final concentration of ethanol (≦0.2%) present in the cell cultures.

### Cells

Human promyelocytic leukemia (HL60) cells were grown in RPMI-1640 medium containing 10% fetal bovine serum. The cells were incubated at 37° C. in the presence of 5% CO₂.

### Assay Conditions

HL60 cells were plated at 1.2×10⁵ cells/ml. Eighteen hours after plating, cells in duplicate were treated with the drug. Four days later, the cells were harvested and a nitro blue tetrazolium reduction assay was performed (Collins et al., 1979; J. Exp. Med. 149:969-974). The percentage of differentiated cells was determined by counting a total of 200 cells and recording the number that contained intracellular black-blue formazan deposits. Verification of differentiation to monocytic cells was determined by measuring phagocytic activity (data not shown).

### In Vitro Transcription Assay

Transcription activity was measured in ROS 17/2.8 (bone) cells that were stably transfected with a 24-hydroxylase (24OHase) gene promoter upstream of a luciferase reporter gene (Arbour et al., 1998). Cells were given a range of doses. Sixteen hours after dosing, the cells were harvested and luciferase activities were measured using a luminometer. RLU=relative luciferase units.

### Intestinal Calcium Transport and Bone Calcium Mobilization

Male, weanling Sprague-Dawley rats were placed on Diet 11 (0.47% Ca) diet+AEK oil for one week followed by Diet 11 (0.02% Ca)+AEK oil for 3 weeks. The rats were then switched to a diet containing 0.47% Ca for one week followed by two weeks on a diet containing 0.02% Ca. Dose administration began during the last week on 0.02% calcium diet. Four consecutive intraperitoneal doses were given approximately 24 hours apart. Twenty-four hours after the last dose, blood was collected from the severed neck and the concentration of serum calcium determined as a measure of bone calcium mobilization. The first 10 cm of the intestine was also collected for intestinal calcium transport analysis using the everted gut sac method.

### Biological Activity Results

(20S)-2-Methylene-19,23,24-trinor-1α-hydroxyvitamin D₃ (SMB) shows slightly lower affinity relative to 1,25(OH)₂D₃ in binding to the recombinant vitamin D receptor as shown in FIG. 1. As shown in FIG. 2, SMB has an EC₅₀ of 40 nM for differentiation of HL-60 cells into monocytes in culture. As shown in FIG. 3, SMB has an EC₅₀ of 8 μM with respect to increasing transcription of the 24-hydroxylase gene. In vivo testing demonstrated that SMB is inactive in releasing bone calcium stores at the doses tested (780 and 7020 pmol), (FIG. 4). However, SMB is active in vivo in increasing intestinal calcium transport. The intestinal calcium transport activity of SMB is approximately 80 times lower than that of the native hormone, i.e., a 7020 pmol dose of SMB elicited a similar increase in the serosal/mucosal calcium ratio as did an 87 pmol dose of the native hormone (FIG. 5). The high VDR binding activity of SMB coupled with its calcemic activity profile (i.e., active in intestine, yet inactive in bone) makes it a candidate for the prevention and treatment of certain types of autoimmune diseases (e.g., multiple sclerosis, lupus, diabetes mellitus, host versus graft reaction, rejection of organ transplants), inflammatory diseases (e.g., rheumatoid arthritis, asthma, or inflammatory bowel diseases), certain types of cancers (e.g., leukemia; colon cancer; breast cancer; prostate cancer); psoriasis and a skin diseases (e.g., wrinkles, lack of adequate skin firmness, lack of adequate dermal hydration, or insufficient sebum secretion), and/or renal osteodystrophy.

(20R)-2-Methylene-19,23,24-trinor-1α-hydroxyvitamin D₃ (RMB) binds the vitamin D receptor with a Kᵢ of 4 nM and thus has lower affinity than that of the native hormone (FIG. 6). The ability of RMB to cause differentiation of HL-60 cells is also less than the native hormone as shown in FIG. 7. Reduced in vitro activity by RMB is seen in the transcription assay compared to the native hormone in increasing transcription of the 24-hydroxylase gene (FIG. 8). Like SMB, in vivo testing shows that RMB is essentially inactive on releasing bone calcium stores at the dosages tested (FIG. 9), but is active in stimulating intestinal calcium transport (FIG. 10). Like SMB, RMB is less active than the native hormone in stimulating intestinal calcium transport, showing significantly less activity at 780 pM than native hormone (0.9 vs. 4.1 in the serosal calcium to mucosal calcium ratio). The reasonably high VDR binding activity of RMB along with its calcemic activity profile makes it a candidate for the prevention and treatment of those diseases for which SMB may be indicated (vide supra). Furthermore, the low activity of RMB in promyelocytic cells and bone cells suggests that RMB may exhibit tissue selective activities. In this regard, RMB may be selective in certain cancer therapies.

### Comparison to Other Compounds

Table 1 shows biological data for the compounds from the present disclosure (SMB and RMB) in comparison to other 2-methylene-1α-hydroxy-19-norvitamin D₃ analogs with non-hydroxylated side chains: 2-methylene-19-nor-(20S)-1α-hydroxytrishomopregnacalciferol (referred to as “2MtrisP” in Table 1) and 2-methylene-19-nor-(20R)-1α-hydroxybishomopregnacalciferol (referred to as “(20R)-2MbisP” in Table 1). The present compounds, SMB and RMB, display surprising and unexpected bioactivity in comparison to the known compounds in a number of respects. For example, despite structural similarities (i.e., a 20S methyl group) and essentially identical vitamin D receptor binding behavior when normalized against the native hormone, SMB is more than an order of magnitude less active than 2MtrisP in causing the differentiation of HL-60 cells, when normalized against the native hormone (i.e., 1.2/0.08≈15). The difference between the biological activity of RMB and (20R)-2 MbisP is even more pronounced, despite each compound possessing a 20R methyl group. In comparison to (20R)-2 MbisP, RMB is approximately 10 times less active in binding to the vitamin D receptor (i.e., 0.49/0.05≈10), approximately 360 times less active in the differentiation of HL-60 cells (i.e., 0.18/0.0005≈360), and more than 16 times less active in hydroxylase transcription (i.e., 0.05/0.003>16).

TABLE 1

| | | | Competitive | | 24OHase |
|---|---|---|---|---|---|
| | | | VDR Binding² | HL-60 | Transcription³ |
| Working | | | (Relative | Differentiation³ | (Relative |
| Example¹ | Where | Side chain | Activity⁴) | (Relative Activity⁴) | Activity⁴) |
| SMB | Present | 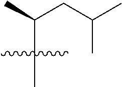 [SMILES: *[C@@H](C)CC(C)C] | 0.4 (0.5) | 40 (0.08) | 8 (0.04) |
| RMB | Present | 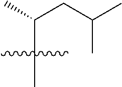 [SMILES: *[C@H](C)CC(C)C] | 4.0 (0.05) | 6000 (0.0005) | 100 (0.003) |
| 2MtrisP | U.S. Pat. No. 7,541,348 | 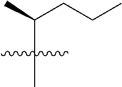 [SMILES: *[C@@H](C)CCC] | 0.075 (0.5) | 1.9 (1.2) | 2.4 (0.06) |
| (20R)-2MbisP | US 2006/0135799 | 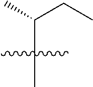 [SMILES: *[C@H](C)CC] | 0.078 (0.49) | 20 (0.18) | 4.2 (0.05) |

¹All compounds are 2-methylene 19-nor compounds.
²Kᵢ, nM.
³EC₅₀, nM.
⁴Activity relative to the native hormone, 1,25(OH)₂D₃, as measured in the same assay.
Relative activity = (value observed for native hormone)/(value observed for working example). Values less than one indicate the working example is less active than the native hormone.

It is understood that the present technology is not limited to the embodiments set forth herein for illustration, but embraces all such forms thereof as come within the scope of the following claims.

What is claimed is:

1. A compound of Formula I or a pharmaceutically acceptable salt thereof 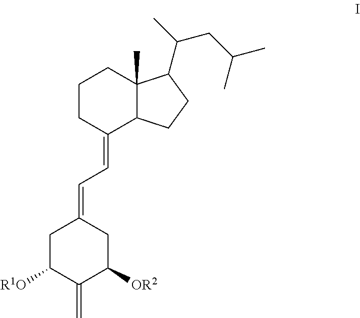
wherein R¹ and R² are independently selected from H and hydroxy protecting groups.

2. The compound of claim 1, wherein R¹ and R² are both hydroxy protecting groups.

3. The compound of claim 2, wherein R¹ and R² are both t-butyldimethylsilyl groups.

4. The compound of claim 1, wherein R¹ and R² are both H and the compound has the Formula II

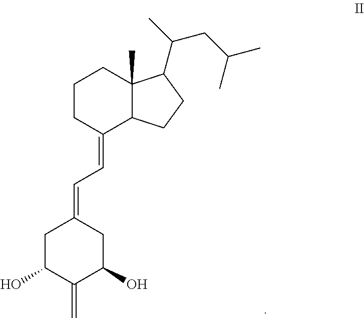 [SMILES: C=C1[C@H](O)CC(=C/C=C2\CCC[C@@]3(C)C2CCC3C(C)CC(C)C)C[C@H]1O]

5. The compound of claim 4, having the Formula IIA

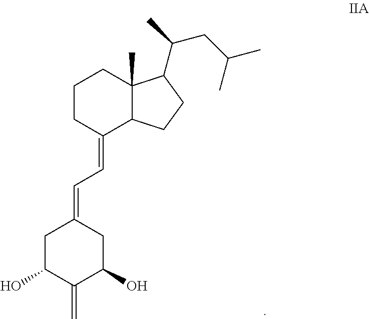 [SMILES: C=C1[C@H](O)CC(=C/C=C2\CCC[C@@]3(C)C2CCC3[C@@H](C)CC(C)C)C[C@H]1O]

6. The compound of claim 5, having the Formula IIIA

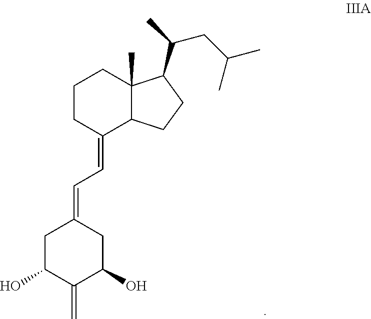 [SMILES: C=C1[C@H](O)CC(=C/C=C2\CCC[C@@]3(C)C2CC[C@@H]3[C@@H](C)CC(C)C)C[C@H]1O]

7. The compound of claim 4, having the Formula IIB

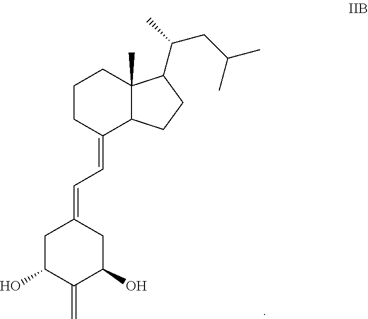 [SMILES: C=C1[C@H](O)CC(=C/C=C2\CCC[C@@]3(C)C2CCC3[C@H](C)CC(C)C)C[C@H]1O]

8. The compound of claim 7, having the Formula IIIB

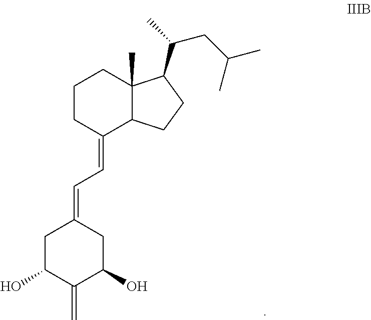 [SMILES: C=C1[C@H](O)CC(=C/C=C2\CCC[C@@]3(C)C2CC[C@@H]3[C@H](C)CC(C)C)C[C@H]1O]

9. A pharmaceutical composition comprising an effective amount of the compound of claim 4 and a pharmaceutically acceptable carrier.

10. The pharmaceutical composition of claim 9, wherein the effective amount comprises from about 0.01 μg to about 1 mg of the compound per gram of the composition.

11. The pharmaceutical composition of claim 9, wherein the effective amount comprises from about 0.1 μg to about 500 μg of the compound per gram of the composition.

12. A method of preventing or treating a biological condition comprising administering an effective amount of the compound of claim 4 or a pharmaceutical composition comprising an effective amount of the compound of claim 4 to a subject, wherein the biological condition is selected from psoriasis; leukemia; colon cancer; breast cancer; prostate cancer; multiple sclerosis; lupus; diabetes mellitus; host versus graft reaction; rejection of organ transplants; an inflammatory disease selected from rheumatoid arthritis, asthma, or inflammatory bowel diseases; a skin condition selected from wrinkles, lack of adequate skin firmness, lack of adequate dermal hydration, or insufficient sebum secretion; or renal osteodystrophy.

13. The method of claim 12, wherein the biological condition is psoriasis.

14. The method of claim 12, wherein the biological condition is renal osteodystrophy.

15. The method of claim 12, wherein the compound or the pharmaceutical composition is administered orally.

16. The method of claim 12, wherein the compound or the pharmaceutical composition is administered parentally.

17. The method of claim 12, wherein the compound or the pharmaceutical composition is administered transdermally or topically.

18. The method of claim 12, wherein the compound or the pharmaceutical composition is administered by delivering the compound or pharmaceutical composition in an aerosol.

19. The method of claim 12, wherein the compound or the pharmaceutical composition is administered in a dosage of from about 0.01 μg per day to about 1 mg per day.

20. The method of claim 12, wherein the compound or the pharmaceutical composition is administered in a dosage of from about 0.1 μg per day to about 500 μg per day.
